SEQUENCE LISTING (13 sequences)

SEQ ID NO: 1 - DNA, 25 bp, Artificial Sequence
  Oligo primer forward
caccgttcagctcggtgtcccgcag

SEQ ID NO: 2 - DNA, 25 bp, Artificial Sequence
  Oligo primer reverse
aaacctgcgggacaccgagctgaac

SEQ ID NO: 3 - DNA, 23 bp, Artificial Sequence
  Krt5
ttcagctcggtgtcccgcagtgg

SEQ ID NO: 4 - DNA, 20 bp, Artificial Sequence
  Krt5_Forward primer
tgccctgccgtttctctact

SEQ ID NO: 5 - DNA, 20 bp, Artificial Sequence
  Krt 6A_Forward primer
ccctctgaacctgcaaatcg

SEQ ID NO: 6 - DNA, 20 bp, Artificial Sequence
  Krt 14_Forward primer
acgagaagatggcggagaag

SEQ ID NO: 7 - DNA, 24 bp, Artificial Sequence
  Krt 17_Forward primer
ggccaggtgggcggcgaaatcaac

SEQ ID NO: 8 - DNA, 20 bp, Artificial Sequence
  Gapdh_Forward primer
catggccttccgtgttccta

SEQ ID NO: 9 - DNA, 20 bp, Artificial Sequence
  Krt 5_Reverse primer
tgatctgctccctctcctca

SEQ ID NO: 10 - DNA, 21 bp, Artificial Sequence
  Krt 6A_Reverse primer
gatctgctccctctcctcagt

SEQ ID NO: 11 - DNA, 24 bp, Artificial Sequence
  Krt 14_Reverse primer
ctctgtcttgctgaagaaccattc

SEQ ID NO: 12 - DNA, 25 bp, Artificial Sequence
  Krt 17_Reverse primer
gagcctggacccttcccgaagtcag

SEQ ID NO: 13 - DNA, 17 bp, Artificial Sequence
  Gapdh_Reverse primer
gcggcacgtcagatcca

(19) United States
(12) Patent Application Publication
(10) Pub. No.: US 2023/0285336 A1
(43) Pub. Date: Sep. 14, 2023

(54) COMPOSITION FOR PREVENTION AND TREATMENT OF SKIN DISEASES CAUSED BY GENETIC MUTATION COMPRISING FERULIC ACID AND ANALOGS THEREOF

(71) Applicants: UNIVERSITY-INDUSTRY COOPERATION GROUP OF KYUNG HEE UNIVERSITY, Yongin-si, Gyeonggi-do (KR); Daegu Gyeongbuk Institute of Science and Technology, Dalseong-gun, Daegu (KR)

(72) Inventors: Hyeung Jin JANG, Seoul (KR); Chang-Hun LEE, Seoul (KR); Won Suk CHUNG, Seoul (KR)

(73) Assignees: UNIVERSITY-INDUSTRY COOPERATION GROUP OF KYUNG HEE UNIVERSITY, Yongin-si, Gyeonggi-do (KR); Daegu Gyeongbuk Institute of Science and Technology, Dalseong-gun, Daegu (KR)

(21) Appl. No.: 17/780,851

(22) PCT Filed: Oct. 5, 2020
(86) PCT No.: PCT/KR2020/013508

§ 371 Date: May 24, 2023

(30) Foreign Application Priority Data
Nov. 28, 2019 (KR) 10-2019-0155901

(51) Int. Cl. A61K 31/192; A61P 17/00

(52) U.S. Cl. CPC A61K 31/192; A61P 17/00

(57) ABSTRACT

The present invention relates to a composition, a cosmetic composition, and a food composition for preventing or treating skin diseases caused by genetic mutation comprising ferulic acid, an analog thereof or a salt thereof.

### TECHNICAL FIELD

The present invention relates to a composition, a cosmetic composition, and a food composition for preventing or treating skin diseases caused by genetic mutation comprising ferulic acid, an analog thereof or a salt thereof.

### BACKGROUND ART

As a primary barrier of the human body, the skin protects the body from temperature and humidity changes, and external environmental stimuli such as ultraviolet rays or pollutants, and plays an important role in maintaining body homeostasis by regulating body temperature. The dermal-epidermal junction of the skin is the part where the dermal layer of the skin joins the epidermal layer of the skin, which not only performs the function of a skin barrier, but also helps the epidermis to firmly adhere to the dermis and to align epidermal cells for wound healing.

On the other hand, the dermal-epidermal junction is the most important factor in genetic skin diseases caused by keratin mutation. As a representative, in the case of epidermolysis bullosa, layers are separated due to lack of protein which is responsible for the adhesion of the epidermis and the dermis, thereby weakening the skin and forming blisters with extreme pain equivalent to third-degree burns caused by minor injury or external pressure.

As a method for treating genetic skin diseases such as epidermolysis bullosa, methods such as modifying the keratin mixture produced in the skin, going through steroid treatment and prescription, and transplanting bone marrow have been studied, but there is still no complete cure for the diseases, and symptomatic therapy for alleviating symptoms or complications is the main treatment method. Among the treatment methods, it is most important to prevent infection of blisters already formed, and painkillers may be used to relieve pain when blisters are formed. Accordingly, there is a demand for developing a novel therapeutic agent capable of fundamentally treating or preventing genetic skin diseases caused by keratin mutation.

### DESCRIPTION OF EMBODIMENTS

### Technical Problem

The technical problem to be solved by the present invention is to provide a pharmaceutical composition for preventing or treating genetic skin diseases comprising ferulic acid, an analog thereof or a pharmaceutically acceptable salt thereof.

Another technical problem to be solved by the present invention is to provide a method for treating genetic skin diseases comprising administering to an individual ferulic acid, an analog thereof or a pharmaceutically acceptable salt thereof.

Yet another technical problem to be solved by the present invention is to provide a cosmetic composition for preventing or improving genetic skin diseases comprising ferulic acid, an analog thereof or a cosmetically acceptable salt thereof.

Still yet another technical problem to be solved by the present invention is to provide a food composition for preventing or improving genetic skin diseases comprising ferulic acid, an analog thereof or a food acceptable salt thereof.

### Technical Solution

According to an aspect of the present invention relates to a composition for treating skin diseases caused by genetic mutation comprising ferulic acid of the following Formula 1, an analog thereof or a pharmaceutically acceptable salt thereof:

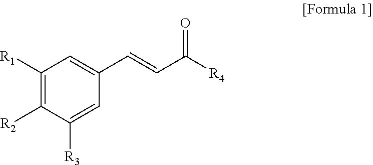

wherein R₁ is H, F, Br, Cl or I;

### R₂ is OH, SH, NH, SeH, F, Br, Cl or I;

R₃ is OCH₃, CH₃, OC₂H₅ or OC₃H₇; and
R₄ is COOH, PO₄, CH₃, NH₃ or NHCOCH₃.

As used herein, “ferulic acid” is a component obtainable from nature, which is mainly contained in plants, and is mainly present in seeds, leaves or skins as a free acid or ester.

The term “analog of ferulic acid” as used herein includes compounds modified within a range that does not significantly change the structure and properties of the parent, such as introduction of functional groups, oxidation, reduction, and substitution of some atoms in the structure of ferulic acid. For example, analogs of ferulic acid include hydroxycinnamic acid such as paracoumarinic acid (p-Coumarinic acid), caffeic acid, sinapinic acid, diferulic acid.

In one embodiment, the Formula 1 may be any one selected from the group consisting of Formulae 2 to 5.

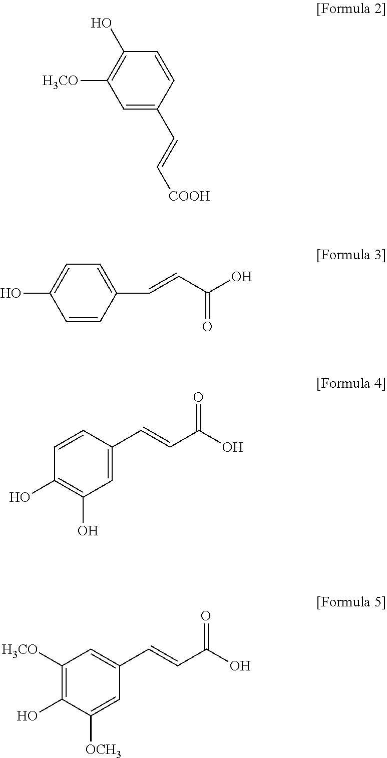 [SMILES: COc1cc(/C=C/C(=O)O)ccc1O ; O=C(O)/C=C/c1ccc(O)cc1 ; O=C(O)/C=C/c1ccc(O)c(O)c1 ; COc1cc(/C=C/C(=O)O)cc(OC)c1O]

The ferulic acid or analog thereof may be chemically synthesized or isolated from natural materials, and may be purchased from the domestic or foreign market and used.

The ferulic acid is known to have a mild antioxidant activity against various active oxygen species derived from oxygen molecules and a strong antioxidant effect against oxidative transition metals. In addition, ferulic acid has an excellent effect of protecting the skin from UV rays by suppressing and/or reducing DNA modification and ROS generation, and is known as a substance helpful for maintaining skin elasticity by inhibiting collagen degradation.

On the other hand, it has not been disclosed or taught that the ferulic acid or analog thereof is a substance effective in preventing, treating and/or improving genetic skin diseases by regulating the amount and/or activity of keratin proteins. Accordingly, the present inventors have identified that ferulic acid may increase the keratin protein expression, and developed a composition for treating or preventing keratin protein-related genetic skin diseases comprising ferulic acid and an analog thereof. Furthermore, the present inventors have confirmed that ferulic acid has the function of regulating the expression of tumor suppressor genes including beta-catenin and c-myc, and accordingly developed a composition wherein ferulic acid and an analog thereof treat or prevent skin cancer, etc., caused by DNA damage.

The term “skin disease caused by genetic mutation” as used herein is a disease caused by mutation in genes directly or indirectly related to skin diseases, in which the expression of keratin genes is suppressed. For example, it includes genetic skin diseases caused by mutation in keratin proteins or skin cancer caused by abnormal regulation of beta-catenin and/or tumor suppressor gene expression due to DNA damage.

In one embodiment, the ferulic acid and analogs thereof may increase the protein expression of keratin 6A (K6A).

The term “keratin” as used herein is proteins which are the main component in various tissues comprising extracellular keratin and intracellular keratin, and are physically tightly coupled by a disulfide bond in body hair (e.g., hair), horns of animals, nails and toenails. In addition, keratin is a type of proteins which form intermediate filaments in cells, and in particular, is important constituent proteins which make keratose in epidermal cells such as skin, etc. According to the characteristics of sequence, keratin includes type-1 keratin such as K10, K14, K16, K17, etc. and type-2 keratin such as KI, K6A, K6B, K5, etc.

The genes encoding the keratin 6A protein may include, but are not limited to, a gene encoding a human keratin 6A protein (e.g., NCBI Accession No. NP_005545), such as KRT6A expressed as NCBI Accession No. NM_005554, etc.

In an exemplary embodiment of the present invention, it was confirmed that when the cross-section of the cultured skin tissue and the skin tissue of an adult mouse was treated with ferulic acid, the expression level of keratin 6A protein increased compared to the control group not treated with ferulic acid (FIGS. 1 to 3), which demonstrates that ferulic acid may increase the expression of keratin 6A protein.

The term “genetic skin disease” as used herein includes all diseases that occur when a mutation occurs in a gene encoding a substance involved in skin growth or maintenance, thereby causing the gene not to function normally, and may be classified into an autosomal dominant genotype and an autosomal recessive genotype.

In one embodiment, the genetic skin disease may be a disease caused by mutation in a gene encoding keratin, and more specifically, may be caused by suppression of the expression of keratin.

In one embodiment, diseases which may be caused by mutation in a gene encoding keratin may be at least one selected from the group consisting of psoriasis, epidermolysis bullosa (EB), Dermatopathia pigmentosa reticularis (DPR), Dowling-Degos Disease (DDD), Epidermolytic Hyperkeratosis (EHK), Bullous Congenital ichthyosiform erythroderma (BCIE), Ichthyosis Bullosa of Siemens (IBS), Palmoplantar keratoderma (PPK), Epidermolytic Palmoplantar keratoderma (EPPK), Pachyonychia congenital, Steatocystoma multiplex and skin cancer. More specifically, the epidermolysis bullosa may be epidermolysis bullosa simplex (EBS), but is not limited thereto, and may include any disease caused by mutation in a gene encoding keratin.

The term “psoriasis” as used herein is a disease forming erythematous papules and plates of various sizes with clear boundaries covered with silvery white scales on the skin, which is a type of chronic inflammatory skin disease characterized by histological hyperproliferation of epidermis. Although the cause is not clearly known, it is known as a type of autoimmune disease where mainly in addition to genetic factors, environmental factors according to living habits act as triggering factors, causing excessive proliferation and immune reaction in the formation of horny cells, so that the immune system mistakes the skin as a foreign substance and attacks it. In the case of psoriasis, as it is known that the keratin 17 expression is up-regulated due to immune system activity including cytokine, it is known that the suppression of keratin 17 expression is related to the reduction and/or treatment of psoriasis.

The term “epidermolysis bullosa (EB)” as used herein refers to a rare genetic disease easily causing blisters even with the slightest wounds accompanied by pain in the skin and mucous membrane, by mutation in a gene making a protein constituting the epidermis, epidermal-dermal junction, and epidermis papillary dermis, and keratin 5 and/or 14 mutations produced in the skin are known to be the main cause of the disease. Epidermolysis bullosa includes epidermolysis bullosa simplex (EBS), junctional epidermolysis bullosa (JEB), and dystrophic epidermolysis bullosa (DEB), and it is known that most epidermolysis bullosa patients have epidermolysis bullosa simplex.

The term “Dermatopathia pigmentosa reticularis (DPR)” as used herein is a rare autosomal dominant congenital disorder which is a form of ectodermal dysplasia. Specifically, keratin 5 and/or keratin 14 mutations produced in the skin are known to be the main cause of the disease. Symptoms include lack of sweat glands, thin hair, brittle nails, stained skin, lack of fingerprints, etc.

The term “Dowling-Degos Disease (DDD)” as used herein is an autosomal dominant congenital disorder of rare hereditary dermatitis, and specifically, keratin 5 and/or keratin 14 mutations are known to be the main cause of the disease. Symptoms include multiple asymptomatic pigmented macules on the flexors, trunk, and extremities.

The term “Epidermolytic Hyperkeratosis (EHK)” as used herein is a skin disease present at birth, and keratin 1 and/or keratin 10 mutations are known to be the main cause of the disease. Newborns with this disease have red skin and severe blisters, and the skin tone in areas such as joints, scalp, or neck appears darker than normal skin tone.

The term “Bullous Congenital ichthyosiform erythroderma (BCIE)” as used herein is a skin disease accompanying erythema, blistering, corrosion, etc. at birth, and keratin 1 and/or keratin 10 mutations are the main cause of the disease.

The term “Ichthyosis Bullosa of Siemens (IBS)” as used herein is an autosomal dominant congenital disorder of rare hereditary dermatitis, specifically caused by keratin 2 mutation. It shows symptoms similar to epidermal keratosis, but unlike epidermal keratosis, it is a disease that affects only the upper layer of the epidermis.

The term “Palmoplantar keratoderma (PPK) or Epidermolytic Palmoplantar keratoderma (EPPK)” as used herein is an autosomal dominant disorder limited to the palms and/or soles of the feet, and specifically it is a disease caused by the mutation of at least one of keratin 1, keratin 9, and keratin 16.

The term “Pachyonychia congenital” as used herein is a disease caused by the mutation of at least one of keratin 6A, keratin 6B, keratin 16 and keratin 17. Symptoms include thick toenails, plantar keratosis, or severe pain in the soles of the feet.

The term “Steatocystoma multiplex” as used herein is a disease caused by keratin 17 mutation, which is a cystic disease found with red, hard, fixed subcutaneous nodules of various sizes spread throughout the neck, back, abdomen and extremities.

The term “skin cancer” as used herein refers to a malignant tumor occurring in the skin, and primary skin malignant tumors include basal cell carcinoma, squamous cell carcinoma and malignant melanoma. The main cause is known to be UV rays of the sunlight damaging DNA and affecting cell growth and differentiation. It has been found out that beta-catenin mutation exists in about 75% of skin cancers, and beta-catenin is known as the first genome to be mutated in the process of forming skin cancer.

In the case of genes encoding keratin, although the expression positions and timings are different, gene duplication frequently occurs, and thus molecular homology is very high.

The keratins having high molecular homology may affect mutual gene expression. In an exemplary embodiment of the present invention, as a result of inducing mutation in the keratin 5 gene, it was confirmed that the mRNA expression of keratin 6A was reduced as well (FIG. 5).

In addition, mutagenic keratin may be replaced by the overexpression of other keratin proteins. For example, the effect of a mutant keratin gene causing pathology may be offset through overexpression of paralogous keratin (Kerns et al, 2007, PNAS).

In an exemplary embodiment of the present invention, as a result of treating epidermal keratinocytes where the keratin 5 gene was knocked down with ferulic acid, it was confirmed that the mRNA expression level of keratin 6A increased (FIG. 6). The above experiments suggest that mutation in keratin 5 gene and keratin 6A protein have high molecular homology, and the symptoms caused by the mutation in one gene may be offset by overexpression of the other gene.

The above results show that the ferulic acid and analogs thereof of the present invention may induce keratin 6A protein expression, and provide a composition for fundamental treatment of various genetic skin diseases resulting from mutation in the gene encoding keratin.

Also specifically, the ferulic acid and analogs thereof may regulate the expression of beta-catenin protein.

The term “beta-catenin” as used herein is a protein involved in adhesion of cells, and control and regulation of gene transcription. Beta-catenin involves in intracellular signal transduction in the Wnt signaling pathway. The mutation and/or overexpression of beta-catenin is known to cause many cancers including skin cancer, hepatocellular carcinoma, and endometrial cancer, etc.

The ferulic acid and analogs thereof may be used to treat skin cancer by regulating the expression of beta-catenin and/or c-myc protein. The ferulic acid and analogs thereof may regulate the expression of beta-catenin in the epidermal layer and the dermal layer of skin cells differently. Specifically, the ferulic acid and analogs thereof may suppress the expression of beta-catenin protein in the epidermal layer of skin cells, while increasing the expression of beta-catenin protein in the dermal layer of skin cells.

Accordingly, the ferulic acid and analogs thereof may regulate the metastasis and penetration of skin cancer cells by controlling the expression of beta-catenin, and accelerate the recovery rate by controlling factors such as beta-catenin and keratin 14 in the wound tissue after treating skin cancer.

In an exemplary embodiment of the present invention, as a result of confirming the expression levels of beta-catenin protein and c-myc protein in skin epidermal stem cells, it was confirmed that the expression of beta-catenin and c-myc proteins was suppressed when treated with ferulic acid (FIGS. 7 and 8).

In an exemplary embodiment of the present invention, as a result of confirming the expression levels of beta-catenin protein and c-myc protein in skin dermal stem cells, it was confirmed that the expression levels of beta-catenin and c-myc proteins increased when treated with ferulic acid (FIGS. 9 and 10).

The above results suggest that the ferulic acid and analogs thereof of the present invention may be effectively utilized in the treatment of skin cancer by penetrating into the dermis and regulating the expression levels differently in the epidermis and dermis.

It may be a composition for treating genetic skin diseases comprising the ferulic acid, an analog thereof or a pharmaceutically acceptable salt thereof.

The term “pharmaceutically acceptable salt” as used herein may be an acid addition salt formed by a free acid.

In the present invention, there is no particular limitation in the pharmaceutically acceptable salt as long as it is commonly used in the art. As specific examples, a salt may be formed by using non-toxic inorganic acids such as hydrochloric acid, hydrobromic acid, sulfonic acid, amidosulfuric acid, phosphoric acid and nitric acid, or non-toxic organic acids such as acetic acid, propionic acid, succinic acid, glycolic acid, stearic acid, lactic acid, tartaric acid, citric acid, paratoluenesulfonic acid and methanesulfonic acid. In addition, examples may include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitro benzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalene-1-sulfonate, naphthalene-2-sulfonate, mandelate, etc.

For administration, the pharmaceutical composition of the present invention may comprise a pharmaceutically acceptable carrier, excipient or diluent in addition to the ferulic acid or analog thereof of the present invention. The carrier, excipient and diluent may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil.

In addition, the pharmaceutical composition of the present invention may be applied in any form of formulation, and more specifically, may be a transdermal permeable formulation. Transdermal permeable formulations may be ointments, gels, creams, sprays, etc., but those skilled in the art may appropriately select and mix them without difficulty according to type, use, and purpose.

In one embodiment, for transdermal administration, ferulic acid or an analog thereof may be mixed with vaseline, and any excipients and additives necessary for transdermal administration may be mixed and used without limitation.

Another aspect of the present invention relates to a method for treating skin diseases caused by genetic mutation, comprising administering to an individual in need of treatment a pharmaceutical composition comprising ferulic acid, an analog thereof or a pharmaceutically acceptable salt thereof.

The term “Ferulic acid,” “analog” and “skin disease caused by genetic mutation” are as described above.

The term “pharmaceutically effective amount” as used herein means an amount sufficient to treat a disease at a reasonable benefit/risk ratio applicable to medical treatment. The effective dose level may be determined by factors including the patient's gender, age, type of disease, severity, drug activity, sensitivity to drugs, administration time, administration route and excretion rate, duration of treatment, concurrent drugs, and other factors well known in the medical field.

The pharmaceutical composition of the present invention may be administered as an individual therapeutic agent or may be administered in combination with other therapeutic agents, and may be administered sequentially or simultaneously with a conventional therapeutic agent. In addition, the pharmaceutical composition of the present invention may be administered once or multiple times. It is important to administer an amount which can obtain the maximum effect with a minimum amount without side effects taking all of the above factors into consideration, and the administration amount may be easily determined by those skilled in the art.

As used herein, the term “individual” includes animals or humans with a genetic skin disease whose symptoms may be improved by administration of the pharmaceutical composition according to the present invention. By administering the therapeutic composition according to the present invention to an individual, it is possible to effectively prevent and treat genetic skin diseases.

As used herein, the term “administration” means introducing a predetermined substance into a human or animal by any suitable method, and as an administration route of the therapeutic composition according to the present invention, the therapeutic composition may be administered orally or parenterally through any general route as long as it can reach the target tissue. In addition, the therapeutic composition according to the present invention may be administered by any device capable of moving the active ingredient to the target cell.

The preferred dosage of the pharmaceutical composition according to the present invention varies depending on the condition and weight of the patient, severity of the disease, drug form, administration route and duration, but may be appropriately selected by those skilled in the art.

Yet another aspect of the present invention relates to a cosmetic composition for preventing or improving skin diseases caused by genetic mutation comprising ferulic acid of the following Formula 1, an analog thereof or a cosmetically acceptable salt thereof.

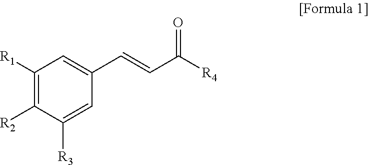

wherein R₁ is H, F, Br, Cl or I;

### R₂ is OH, SH, NH, SeH, F, Br, Cl or I;

R₃ is OCH₃, CH₃, OC₂H₅ or OC₃H₇; and
R₄ is COOH, PO₄, CH₃, NH₃ or NHCOCH₃.

In one embodiment, the Formula 1 may be any one selected from the group consisting of Formulae 2 to 5.

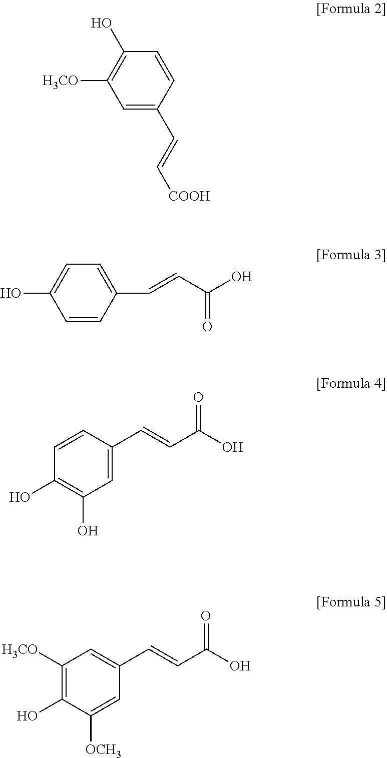 [SMILES: COc1cc(/C=C/C(=O)O)ccc1O ; O=C(O)/C=C/c1ccc(O)cc1 ; O=C(O)/C=C/c1ccc(O)c(O)c1 ; COc1cc(/C=C/C(=O)O)cc(OC)c1O]

The term “Ferulic acid,” “analog” and “skin disease caused by genetic mutation” are as described above.

In an exemplary embodiment of the present invention, as a result of inducing mutation in the keratin 5 gene, it was confirmed that the mRNA expression of keratin 6A was reduced as well (FIG. 5).

In an exemplary embodiment of the present invention, as a result of treating epidermal keratinocytes in which the keratin 5 gene was knocked down with ferulic acid, it was confirmed that the mRNA expression level of keratin 6A increased (FIG. 6). The above experiments suggest that mutation in keratin 5 gene and keratin 6A protein have high molecular homology, and the symptoms caused by the mutation in one gene may be offset by overexpression of the other gene.

The above results suggest that the ferulic acid of the present invention may induce keratin 6A protein expression, and may be effective in improving various genetic skin diseases resulting from mutation in the gene encoding keratin, and thus the ferulic acid and analogs thereof may be utilized in cosmetic compositions.

The cosmetic composition of the present invention may be prepared in a formulation selected from the group consisting of a solution, external ointment, cream, foam, nourishing skin lotion, flexible skin lotion, pack, soft water, emulsion, makeup base, essence, soap, liquid detergent, bath agent, sunscreen cream, sun oil, suspension, emulsion liquid, paste, gel, lotion, powder, soap, surfactant-containing cleansing, oil, powder foundation, emulsion foundation, wax foundation, patch and spray, but is not limited thereto.

The cosmetic composition of the present invention may further comprise one or more cosmetically acceptable carriers to be mixed with general skin cosmetics, and as common ingredients, for example, oil, water, a surfactant, a humectant, a lower alcohol, a thickener, a chelating agent, a colorant, a preservative, a fragrance, etc. may be properly mixed, but the ingredients are not limited thereto.

The cosmetically acceptable carrier included in the cosmetic composition of the present invention varies depending on the formulation of the cosmetic composition.

When the formulation of the present invention is an ointment, paste, cream or gel, animal oil, vegetable oil, wax, paraffin, starch, tragacanth, cellulose derivative, polyethylene glycol, silicone, bentonite, silica, talc, zinc oxide, etc. may be used as an ingredient of the carrier, but the ingredient is not limited thereto. The above may be used alone or two or more may be used in combination.

When the formulation of the present invention is a powder or a spray, lactose, talc, silica, aluminum hydroxide, calcium silicate, polyamide powder, etc. may be used as an ingredient of the carrier, and in particular, in the case of a spray, additional propellants such as chlorofluoro hydrocarbon, propane/butane or dimethyl ether may be additionally included, but the ingredient is not limited thereto. The above may be used alone or two or more may be used in combination.

When the formulation of the present invention is a solution or emulsion liquid, a solvent, solubilizer or emulsifier may be used as an ingredient of the carrier. For example, water, ethanol, isopropanol, ethyl carbonate, ethyl acetate, benzyl alcohol, benzyl benzoate, propylene glycol, 1,3-butyl glycol oil, etc. may be used. In particular, cottonseed oil, peanut oil, corn germ oil, olive oil, castor oil and sesame oil, jojoba oil, glycerol aliphatic ester, polyethylene glycol or sorbitan fatty acid ester may be used, but the ingredient is not limited thereto. The above may be used alone or two or more may be used in combination.

When the formulation of the present invention is a suspension, a liquid diluent such as water, ethanol or propylene glycol, a suspending agent such as ethoxylated isostearyl alcohol, polyoxyethylene sorbitol ester and polyoxyethylene sorbitan ester, crystalline cellulose, aluminum metahydroxide, bentonite, agar or tragacanth, etc. may be used as an ingredient of the carrier, but the ingredient is not limited thereto. The above may be used alone or two or more may be used in combination.

In addition, the composition of the present invention may be used by a transdermal administration method such as directly applying or spraying to the skin, and the composition of the present invention may be administered through any general route as long as it can reach the target tissue.

The usage amount of the composition of the present invention may be appropriately adjusted according to individual differences such as age, degree of lesion, etc. or formulations, and may be used by applying an appropriate amount to the skin once to several times a day for one week to several months.

Another aspect of the present invention relates to a food composition for preventing or improving skin diseases caused by genetic mutation comprising ferulic acid of the following Formula 1, an analog thereof or a food acceptable salt thereof.

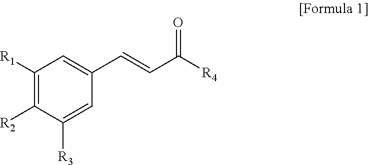

wherein R₁ is H, F, Br, Cl or I;

### R₂ is OH, SH, NH, SeH, F, Br, Cl or I;

R₃ is OCH₃, CH₃, OC₂H₅ or OC₃H₇; and
R₄ is COOH, PO₄, CH₃, NH₃ or NHCOCH₃.

In one embodiment, the Formula 1 may be any one selected from the group consisting of Formulae 2 to 5.

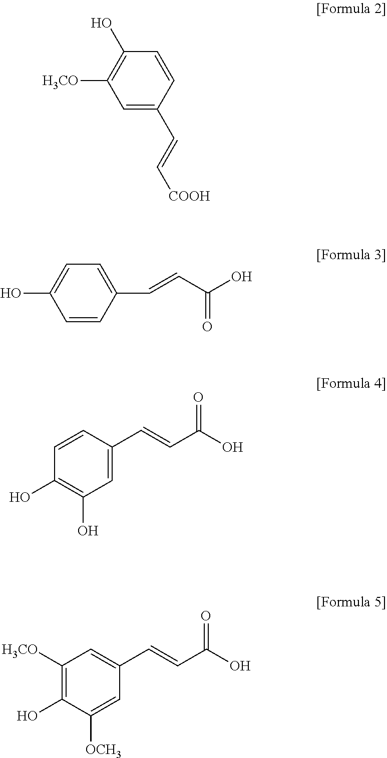 [SMILES: COc1cc(/C=C/C(=O)O)ccc1O ; O=C(O)/C=C/c1ccc(O)cc1 ; O=C(O)/C=C/c1ccc(O)c(O)c1 ; COc1cc(/C=C/C(=O)O)cc(OC)c1O]

The term “Ferulic acid,” “analog” and “skin disease caused by genetic mutation” are as described above.

The term “food composition” as used herein may be exemplified by meat, snacks, dairy products, beverages, etc., but is not limited thereto, and may be understood as a concept including all typical health functional food.

Food to which ferulic acid or analog thereof of the present invention may be added includes meat, bread, sausage, chocolates, snacks, candies, confectionery, ramen, pizza, other noodles, gum, dairy products including ice cream, various soups, beverages, tea, vitamin complex, health functional supplements, etc.

The type of food may specifically be health functional food. The health functional food may include various nutrients, vitamins, minerals (electrolytes), flavoring agents such as synthetic flavoring agents and natural flavoring agents, coloring agents and enhancers (cheese, chocolate, etc.), pectic acid and salts thereof, organic acids, protective colloidal thickeners, pH regulators, stabilizers, preservatives, glycerin, alcohol, carbonation agents used in carbonated beverages, etc. These ingredients may be used alone or in combination, and the proportion of these additives is generally selected from a range of 0.001 to 50 parts by weight based on the total weight of the composition.

The health functional food is a food emphasizing the bioregulatory function of food, with added value to act and express for a specific purpose using a physical, biochemical, and bioengineering method. The ingredients of these health functional food are designed and processed to sufficiently exert body control functions related to biophylaxis, regulation of body rhythm, prevention and recovery of diseases, and may comprise food supplementary additives, sweeteners or functional raw materials which are acceptable as food.

The health functional food may be any one formulation selected from the group consisting of tablets, granules, powder, capsules, liquid solutions and pills for the purpose of improving genetic skin diseases, but is not limited thereto. In one embodiment, the health functional food in the form of tablets may be prepared by granulating a mixture of ferulic acid or an analog thereof, an excipient, a binder, a disintegrant and other additives in a conventional manner, and then adding a lubricant, etc. thereto and compress-molding the same, or directly compress-molding the mixture. In addition, the health functional food in the form of tablets may comprise a bittering agent, etc., if necessary, and may be coated with a suitable coating agent, if necessary.

Among the health functional food in the form of capsules, hard capsules may be prepared by filling a conventional hard capsule with a mixture of ferulic acid or an analog thereof and an additive such as an excipient, or granules thereof or coated granules. Soft capsules may be prepared by filling a capsule base such as gelatin with a mixture of ferulic acid or an analog thereof and an additive such as an excipient. The soft capsule may comprise a plasticizer such as glycerin or sorbitol, a colorant, a preservative, etc., if necessary.

The health functional food in the form of pills may be prepared by molding a mixture of ferulic acid or an analog thereof, an excipient, a binder, a disintegrant, etc. in an appropriate manner, and, if necessary, coating the skin with white sugar or other suitable coating agents, or applying powers with starch, talc or any suitable substance.

The health functional food in the form of granules may be prepared by making a mixture of ferulic acid or an analog thereof, an excipient, a binder, a disintegrant, etc. in a granular form by any suitable method, and may comprise a flavoring agent, a bittering agent, etc., if necessary.

Definitions of terms such as the excipient, binder, disintegrant, lubricant, bittering agent, flavoring agent, etc. are as known in the art and may include the same or similar ones for their functions.

In addition, the type of the food may be a food additive. The food additive means a substance used by methods such as addition, mixture, infiltration to food for manufacturing, processing, or preserving food. The food additive may include natural products and synthetic products, and may be classified according to function and use. Currently, about 370 kinds of chemically synthesized products and 50 kinds of natural additives are approved as food additives in Korea. Mainly according to use, food additives are classified and used as preservatives, disinfectants, antioxidants, coloring agents, color fixing agents, bleaching agents, seasonings, sweeteners, flavoring agents, expanding agents, strengthening agents, improving agents, emulsifiers, thickeners (thickening agents) and stabilizers, film agents, gum base agents, defoaming agents, solvents, release agents, insect repellents, quality improvers, and other additives for food manufacturing.

The form of the food additive may include powder, granule, tablet, capsule or liquid form, and specifically may be in the form of a capsule, but is not limited thereto.

When the ferulic acid or analog thereof of the present invention is used as a food composition, the ferulic acid or analog thereof may be added as it is or used with other food or food ingredients, and may be appropriately used according to a conventional method. The mixing amount of the ferulic acid or analog thereof may be appropriately determined according to the purpose of its use (prevention, health or improvement, therapeutic treatment).

### Advantageous Effects of Invention

The ferulic acid or analog thereof of the present invention is a component contained in natural substances, and the composition comprising the ferulic acid or analog thereof induces the expression of keratin protein and may be widely used for treatment, prevention and improvement of genetic skin diseases.

The effects of the present invention are not limited to the above-mentioned effects, and it should be understood that the effects of the present invention include all effects that could be inferred from the configuration of the invention described in the detailed description of the invention or the appended claims.

### BRIEF DESCRIPTION OF THE DRAWINGS

FIG. 1 shows the results of confirming the amount of keratin 6A protein through immunofluorescence staining after treating the cultured skin tissue with ferulic acid;

FIG. 2 shows the results of confirming the amount of keratin 6A protein through Western blotting after treating the skin tissue of an adult mouse with ferulic acid;

FIG. 3 shows the results of confirming the amount of keratin 6A protein through immunofluorescence staining after treating the skin tissue of an adult mouse with ferulic acid;

FIG. 4 shows the result of confirming whether keratin 5 is knocked down in the cell;

FIG. 5 shows the results of confirming the expression levels of keratin 5 and keratin 6A mRNA through qPCR in the keratin 5 knock-down cells;

FIG. 6 shows the results of confirming through Western blotting whether the expression of keratin 6A protein increased by treating the keratin 5 knock-down cells with ferulic acid;

FIG. 7 shows the results of confirming the amount of beta-catenin protein through immunofluorescence staining after treating the cultured skin epidermal stem cells with ferulic acid;

FIG. 8 shows the results of confirming the amount of beta-catenin protein through Western blotting after treating the cultured skin epidermal stem cells with ferulic acid;

FIG. 9 shows the results of confirming the amount of beta-catenin protein through immunofluorescence staining after treating the cultured skin dermal stem cells with ferulic acid; and

FIG. 10 shows the results of confirming the amount of beta-catenin protein through Western blotting after treating the cultured skin dermal stem cells with ferulic acid.

### DETAILED DESCRIPTION OF EXEMPLARY EMBODIMENTS

Hereinafter, the present invention will be described in detail by way of examples. However, the following examples only illustrate the present invention, and the present invention is not limited to the following examples.

### Example 1. Confirmation of the Expression Level of Keratin 6A Protein after Treating the Skin Tissue of Mouse with Ferulic Acid

In order to confirm whether ferulic acid induces the expression of keratin 6A protein from the skin tissue of a mouse, the cross-sections of the cultured skin tissue and the skin tissue of an adult mouse were treated with ferulic acid to confirm the expression level of keratin 6A protein using Western blotting and immunofluorescence staining.

First, after peeling off the skin of a mouse within one day of age, a circular skin piece was spread on a 24-well plate using a 4 mm punch (Kai industries). The cell medium was carefully added with the growth factor Cnt-57 according to the method of CELLnTEC, and the medium was replaced once every two days. For the skin tissue culture method, non-patent literature Mazzalupo et al., J Invest Dermatol, 2002 may be referred to. After culturing the group treated with ferulic acid and the untreated control group for 12 days, the expression of keratin 6A protein in the skin cells was confirmed using immunofluorescence staining.

Specifically, for cell fluorescence staining, 500 μl of a 4% paraformaldehyde solution (solvent, PBS) was reacted for 20 minutes, and then washed three times with a washing buffer (0.1 M, PBS). Then, a primary antibody keratin 6A (rabbit anti-keratin 6A, 1:200, Covans, NJ, USA) was reacted for 15 hours. After washing three times with a washing buffer (0.1 M, PBS), a secondary antibody Alexa 488 (anti-rabbit IgG extracted from donkey, 1:1000, Invitrogen, CA, USA) was reacted for 1 hour. Then, after washing three times with a washing buffer (0.1 M, PBS), it was reacted with a DAPI (4′,6-diamidino-2-phenylindole, 1:1000, Invitrogen, CA, USA) solution for 3 minutes. After washing again three times with a washing buffer (0.1 M, PBS), fluorescent photos were taken using a FluoView FV1000 confocal microscope (Olympus, Tokyo, Japan).

For tissue fluorescence staining, after removing paraffin from the paraffin-coated skin tissue, the tissue was washed three times with a washing buffer (0.1 M, PBS), and reacted with a solution containing hydrogen peroxide (0.1 M PBS, 0.2% H₂O₂) for 30 minutes to eliminate the activity of endogenous peroxidase. The tissue was reacted with a blocking solution (5% bovine serum, 0.1% Triton X-100) for 2 hours, and washed three times with a washing buffer (0.1 M, PBS). Then, primary antibodies keratin 6A (rabbit anti-keratin 6A, 1:200, Covans, NJ, USA) and beta-catenin (anti-beta catenin extracted from mouse, 1:200, Invitrogen, CA, USA) were reacted for 15 hours. After washing three times with a washing buffer (0.1 M, PBS), secondary antibodies Alexa 488 (anti-rabbit IgG extracted from donkey, 1:1000, Invitrogen, CA, USA) and Alexa 647 (anti-mouse IgG extracted from goat, 1:1000, Invitrogen, CA, USA) were reacted for 1 hour. After washing three times with a washing buffer (0.1 M, PBS), it was reacted with a DAPI (1:1000, Invitrogen, CA, USA) solution for 3 minutes. Then, after washing three times with a washing buffer (0.1 M, PBS), fluorescence photos were taken using a FluoView FV1000 confocal microscope (Olympus, Tokyo, Japan).

In addition, the expression level of protein was confirmed using Western blotting.

First, in order to confirm the expression levels of two proteins, keratin 6A and beta-catenin, skin tissue was collected and protein was extracted using a dissolution buffer (pH 7.4, 50 mM, Tris-HCL, 150 mM NaCL, 1 mM EDTA, 1 mM EGTA, 10 mg/mL aprotinin, 10 mg/mL leupeptin, 5 mM phenylmethylsulfonyl fluoride and 1 mM DTT). The extracted protein was quantified using a Bradford reaction solution (Bio-Rad, CA, USA), and 20 μL of the protein was electrophoresed by 10% SDS-PAGE, and transferred to a nitrocellulose membrane (Merck, MA, USA). The membrane was reacted with primary antibodies keratin 6A (rabbit anti-keratin 6A, 1:200, Covans, NJ, USA) and β-actin (anti-β-actin extracted from mouse, 1:1000, Santa Cruz, TX, USA), and washed three times with TBS-T (Tris Buffered Saline with Tween 20) for 10 minutes, and then the membrane was reacted with a secondary antibody (Santa Cruz, TX, USA) for 1 hour. Analysis on the expression of the antibodies was performed using enhanced chemiluminescence (ECL) expressed by a horseradish peroxidase (HRP)-linked secondary antibody and a chemidoc imaging system (BioRed, CA, USA).

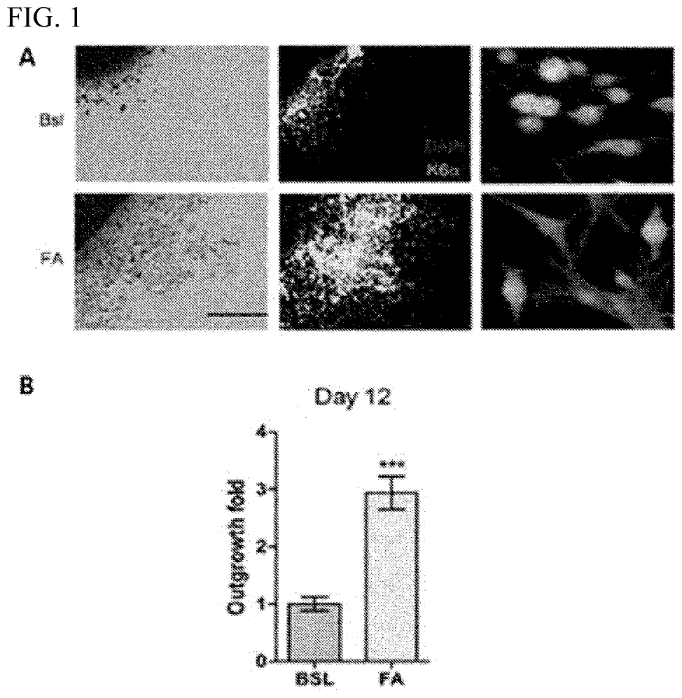

As a result, it was confirmed that the expression level of keratin 6A protein increased in the skin tissue treated with ferulic acid compared to the control group not treated with ferulic acid (FIG. 1).

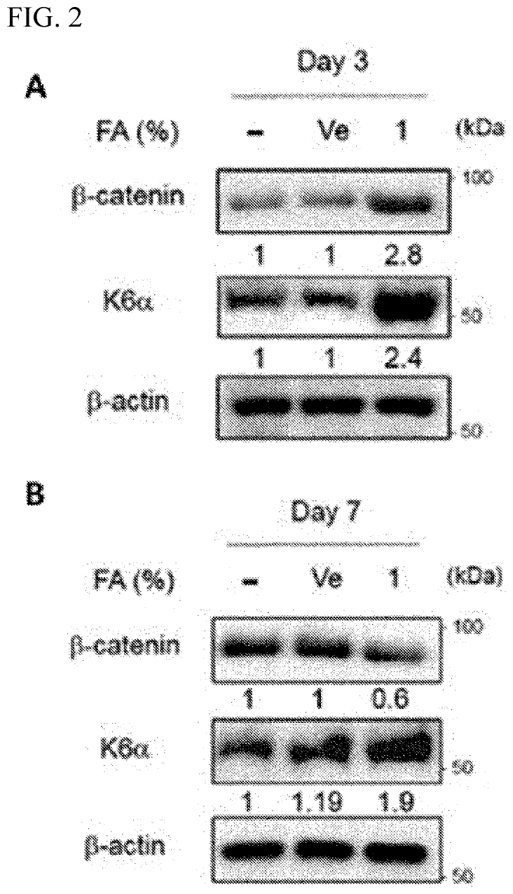

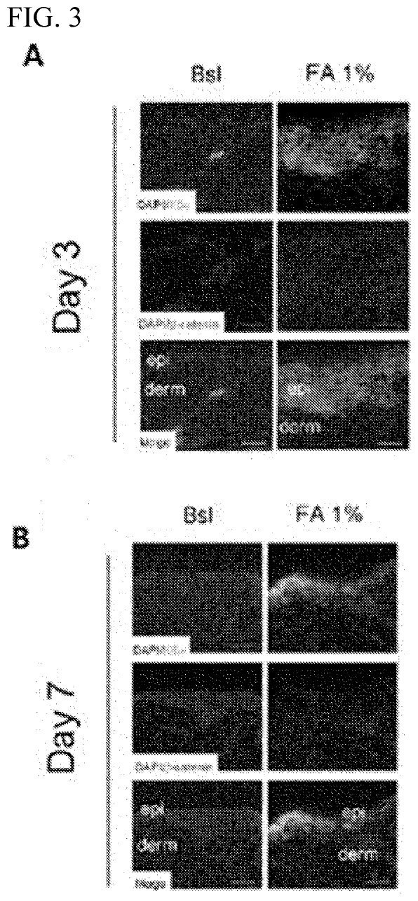

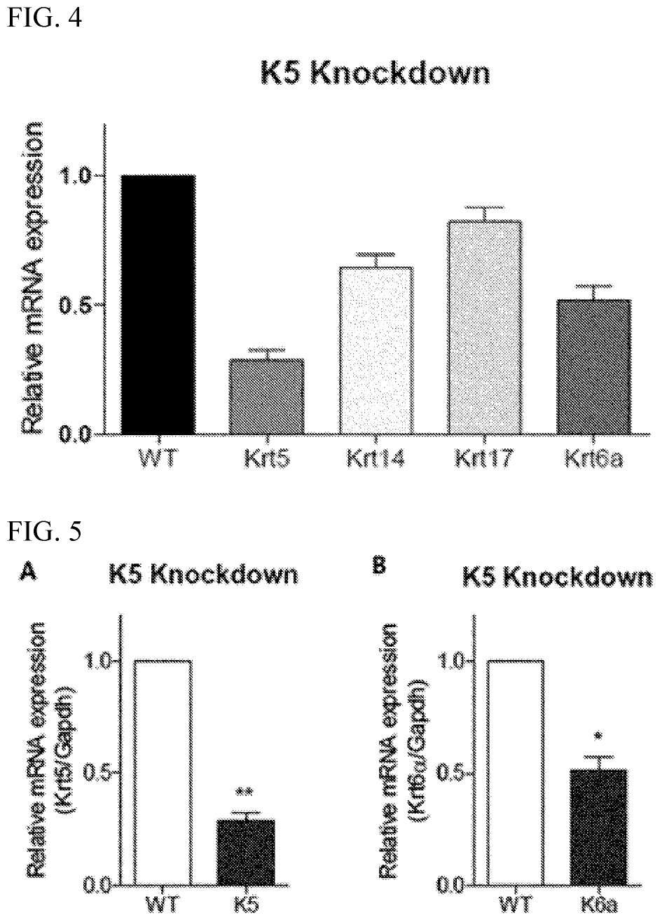

In addition, as a result of confirming the expression level of keratin 6A protein after treating the skin tissue of an adult mouse with ferulic acid for 3 days and 7 days, it was confirmed that the expression level of keratin 6A protein increased significantly. Specifically, it was confirmed that the expression level of keratin 6A protein was particularly high 3 days after treating the skin tissue of an adult mouse with ferulic acid (FIGS. 2 and 3).

The above results suggest that ferulic acid is a substance capable of inducing the expression of keratin 6A protein in skin tissue.

### Example 2. Confirmation of the Expression Level of Keratin 6A Protein in Keratin 5 Knock-Down Epidermal Keratinocytes

In order to determine whether the reduction in the expression of keratin 5 protein affects the expression of keratin 6A protein, the expression level of keratin 6A protein was confirmed after knocking down the gene encoding keratin 5 in kera308 cells, which are epidermal keratinocytes.

### 2-1. Preparation of Kera308 Cell

Kera308 (Cell line service, Germany, #400429) was cultured in a high glucose DMEM medium supplemented with 10% (v/v) fetal bovine serum (FBS). Cells were cultured in an incubator at 37° C. under a 5% CO₂ condition. In order to find a gRNA capable of knocking out a specific gene with high efficiency, as will be described in Examples 2-2 and 2-3 below, as sgRNA and Cas9 protein, 1 μg of in vitro transcribed sgRNA and 4 μg of Cas9 protein (Toolgen) were introduced in the cultured 1×10⁵ Kera308 cells using Lipofectamine 2000 (ThermoFisher Scientific, New York, USA) (in vitro).

2-2. Kera308 Cell Transfection: sgRNA Design

CRISPR/Cas9 target site selection and putative off-target testing of keratin 5 (NCBI Accession No. NM_027011.3) was performed using CRISPR RGEN Tools (htto://www.rgenome.net/cas-designer). As the CRISPR/Cas9 target site, DNA sequences without 0-, 1-, or 2 bp mismatch sites were selected as sgRNA target sites except for the on-target sequence site.

2-3. Kera308 Cell Transfection: sgRNA Synthesis

Two complementary oligonucleotides were annealed and extended to PCR-amplify the templates for sgRNA synthesis. The target site sequence used at this time, the primer sequence for amplifying the same, and the DNA target sequence (including PAM) targeted by the sgRNA obtained therefrom are summarized in Table 1.

TABLE 1

| SEQ | | |
|---|---|---|
| ID | | |
| NO. | | Sequence |
| | Primer | |
| | sequence | |
| 1 | Oligo primer | 5′-caccgTTCAGCTCGGTGTCCCGCAG-3′ |
| | forward | |
| 2 | Oligo primer | 5′-aaacCTGCGGGACACCGAGCTGAAc-3′ |
| | reverse | |
| | DNA target | |
| | sequence | |
| | (including | |
| | PAM sequence) | |
| 3 | Krt5 | 5′-TTCAGCTCGGTGTCCCGCAGTGG-3′ |

### 2-4 Confirmation of the Expression Levels of Keratin 5 and Keratin 6A in Kera308 Cells

In order to confirm whether the gene encoding keratin 5 was knocked down in the cell, the gene expression pattern was investigated by Real-time PCR. In addition, in order to confirm whether the knock-down of the gene encoding keratin 5 affects the expression of the gene encoding keratin 6A, it was confirmed whether the expression of keratin 6A gene was improved using Real-time PCR. mRNA levels were normalized to GADPH, and a wild-type comprising a normal keratin 5 gene was set as a control group.

First, RNA was extracted from a mouse cell line kera308 using an RNA-isolation kit, Hybrid® (Jinol, Seoul, Korea). 1 μg RNA and HyperScript kit (Jinol, Seoul, Korea) were used to synthesize cDNA from the extracted RNA. Then, using the synthesized cDNA, SYBR and target gene (keratin 5, keratin 6A, keratin 14, keratin 17) primers were mixed according to the guideline procedure of Real-time PCR (Applied Biosystems, CA, USA) and put in 96 wells. Then, the plate was inserted into a Real-time PCR machine. The primer sequences of the target genes are as follows.

TABLE 2

| SEQ | | |
|---|---|---|
| ID | Target | |
| NO. | gene | |
| | | Forward primer |
| 4 | Krt 5 | 5′-TGCCCTGCCGTTTCTCTACT-3′ |
| 5 | Krt 6A | 5′-CCCTCTGAACCTGCAAATCG-3′ |
| 6 | Krt 14 | 5′-ACGAGAAGATGGCGGAGAAG-3′ |
| 7 | Krt 17 | 5′-GGCCAGGTGGGCGGCGAAATCAAC-3′ |
| 8 | Gapdh | 5′-CATGGCCTTCCGTGTTCCTA-3′ |
| | | Reverse primer |
| 9 | Krt 5 | 5′-TGATCTGCTCCCTCTCCTCA-3′ |
| 10 | Krt 6A | 5′-GATCTGCTCCCTCTCCTCAGT-3′ |
| 11 | Krt 14 | 5′-CTCTGTCTTGCTGAAGAACCATTC-3′ |
| 12 | Krt 17 | 5′-GAGCCTGGACCCTTCCCGAAGTCAG-3′ |
| 13 | Gapdh | 5′-GCGGCACGTCAGATCCA-3′ |

As a result, it was confirmed that the mRNA expression level of keratin 5 was reduced, which means that the keratin 5 gene was normally knocked down.

In addition, as a result of confirming the mRNA expression level of keratin 6A, which has high molecular homology with keratin 5, it was confirmed that the mRNA expression level was reduced by about 50% in cells in which the gene encoding keratin 5 was knocked down compared to cells in which it was not (FIG. 5).

The above results suggest that keratins with high molecular homology may affect mutual gene expression.

### Example 3. Confirmation of the Expression Level of Keratin 6A Protein after Treating the Keratin 5 Knock-Down Cell with Ferulic Acid

In order to confirm whether ferulic acid affects the expression of keratin 6A protein, after treating kera308 cells with ferulic acid, the amount of keratin 6A protein was confirmed using Western blotting.

The cells were precultured for 15 hours after attaching 1×10⁶ cells/well of kera308 cells, and cultured for 24 hours after being treated with 100 μM ferulic acid. The harvested cells were centrifuged at 13,000 rpm for 5 minutes to take the supernatant, and then the protein concentration was quantified using Bradford assay (BioRed). For Western blotting, refer to the method of Example 1 above.

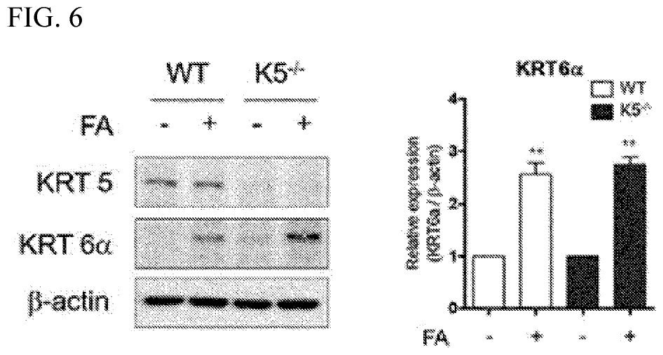

As a result, it was confirmed that when ferulic acid was treated, the expression level of keratin 6A protein increased significantly compared to that not treated with ferulic acid (FIG. 6).

The above results suggest that ferulic acid is a substance capable of increasing the expression level of keratin 6A protein, and thus may be widely used for treating genetic skin diseases caused by keratin 6A mutations or other keratin mutations having high molecular homology with keratin 6A.

### Example 4. Confirmation of the Expression Level of Beta-Catenin Protein after Treating the Skin Tissue of a Mouse with Ferulic Acid

In order to confirm whether ferulic acid regulates beta-catenin protein, an experiment was carried out to confirm the expression levels of beta-catenin protein and c-myc protein in epidermal cells and dermal cells of the skin.

### 4-1. Confirmation of Beta-Catenin Protein Suppression Effect in Skin Epidermal Stem Cells

The expression levels of beta-catenin protein and c-myc protein after treating skin epidermal stem cells with ferulic acid, and the expression levels of beta-catenin protein and c-myc protein after treating skin epidermal stem cells with ferulic acid together with LiCl, which is a beta-catenin protein inducer, were confirmed using immunofluorescence staining and Western blotting.

First, for immunofluorescence staining, the cell fluorescence staining method of Example 1 was referred to. At this time, beta-catenin (anti-beta-catenin extracted from mouse, 1:200, Invitrogen, CA, USA) was used instead of keratin 6A as a primary antibody, and Alexa 647 (anti-mouse IgG derived from goat, 1:1000, Invitrogen, CA, USA) was used instead of Alexa 488 (anti-rabbit IgG extracted from donkey, 1:1000, Invitrogen, CA, USA) as a secondary antibody.

In addition, for Western blotting, the Western blotting method of Example 1 was referred to. In this case, beta-catenin (anti-beta catenin extracted from mouse, 1:200, Invitrogen, CA, USA) was used instead of keratin 6A as a primary antibody.

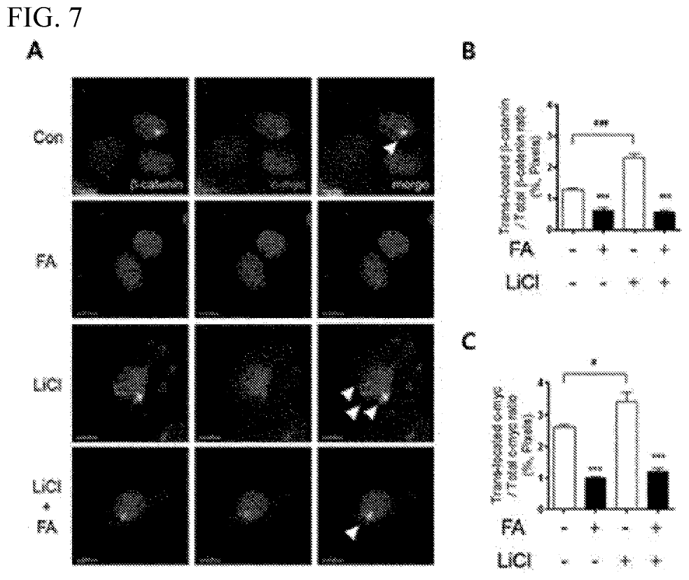

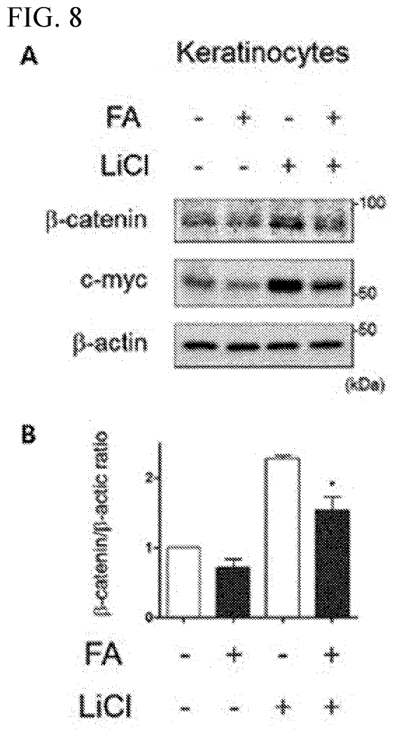

As a result, as shown in FIGS. 7 and 8, it was confirmed that the expressions of beta-catenin and c-myc protein were both suppressed in the skin epidermal stem cells treated with ferulic acid.

### 4-2. Confirmation of Beta-Catenin Protein Suppression Effect in Skin Dermal Stem Cells

The expression levels of beta-catenin protein and c-myc protein after treating skin dermal stem cells with ferulic acid, and the expression levels of beta-catenin protein and c-myc protein after treating skin dermal stem cells with ferulic acid together with LiCl, which is a beta-catenin protein inducer, were confirmed using immunofluorescence staining and Western blotting.

First, for immunofluorescence staining, the cell fluorescence staining method of Example 1 was referred to. At this time, beta-catenin (anti-beta-catenin extracted from mouse, 1:200, Invitrogen, CA, USA) was used instead of keratin 6A as a primary antibody, and Alexa 647 (anti-mouse IgG derived from goat, 1:1000, Invitrogen, CA, USA) was used instead of Alexa 488 (anti-rabbit IgG extracted from donkey, 1:1000, Invitrogen, CA, USA) as a secondary antibody.

In addition, for Western blotting, the Western blotting method of Example 1 was referred to. In this case, beta-catenin (anti-beta catenin extracted from mouse, 1:200, Invitrogen, CA, USA) was used instead of keratin 6A as a primary antibody.

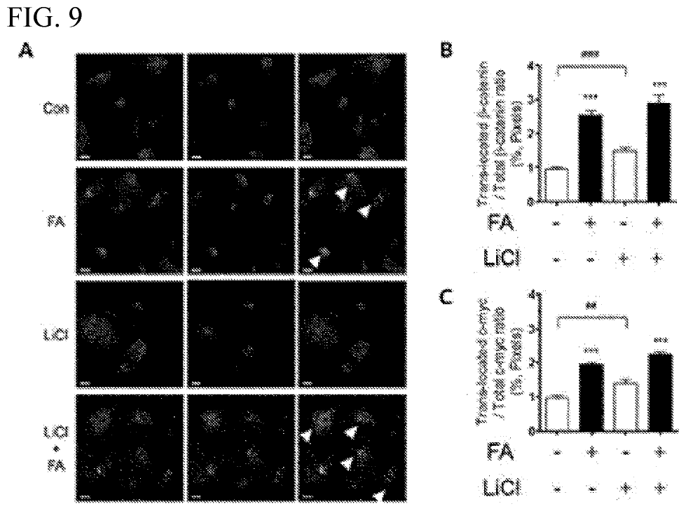

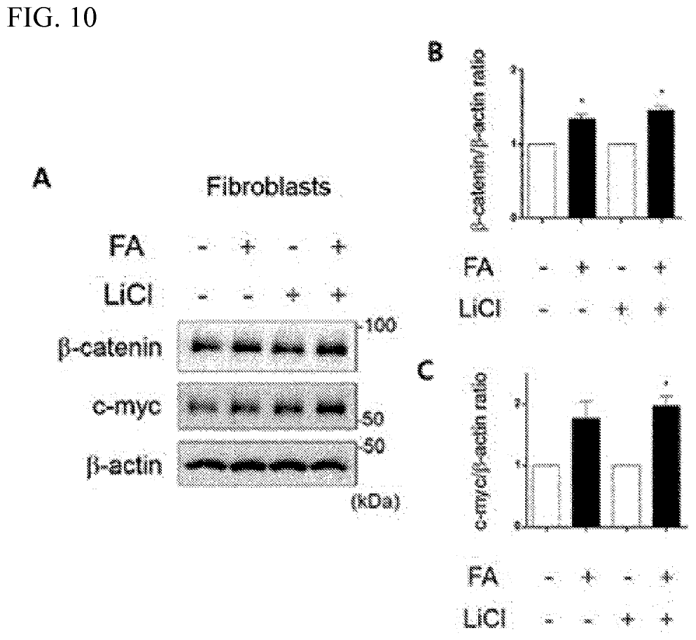

As a result, as shown in FIGS. 9 and 10, it was confirmed that the amounts of beta-catenin and c-myc proteins increased in the skin dermal stem cells.

The above results suggest that ferulic acid is a component capable of regulating beta-catenin protein in skin tissues, has a small size that can go down to the dermis layer when applied to the skin, and may be used in the treatment of skin cancer by regulating the expression of beta-catenin differently in the epidermis and dermis.

The foregoing description of the present invention has been presented for illustrative purposes, and it is apparent to a person having ordinary skill in the art that the present invention can be easily modified into other detailed forms without changing the technical idea or essential features of the present invention. Therefore, it should be understood that the foregoing embodiments are by way of example only, and are not intended to limit the present disclosure. For example, each component which has been described as a unitary part can be implemented as distributed parts. Likewise, each component which has been described as distributed parts can also be implemented as a combined part.

The scope of the present invention is presented by the accompanying claims, and it should be understood that all changes or modifications derived from the definitions and scopes of the claims and their equivalents fall within the scope of the present invention.

What is claimed is:

1. A pharmaceutical composition for preventing or treating skin diseases caused by genetic mutation comprising ferulic acid of the following Formula 1, an analog thereof or a pharmaceutically acceptable salt thereof: 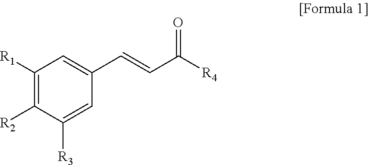
wherein R₁ is H, F, Br, Cl or I;
R₂ is OH, SH, NH, SeH, F, Br, Cl or I;
R₃ is OCH₃, CH₃, OC₂H₅ or OC₃H₇; and
R₄ is COOH, PO₄, CH₃, NH₃ or NHCOCH₃.

2. The pharmaceutical composition of claim 1, wherein a compound represented by the Formula 1 is at least one selected from the group consisting of the following Formulae 2 to 5:

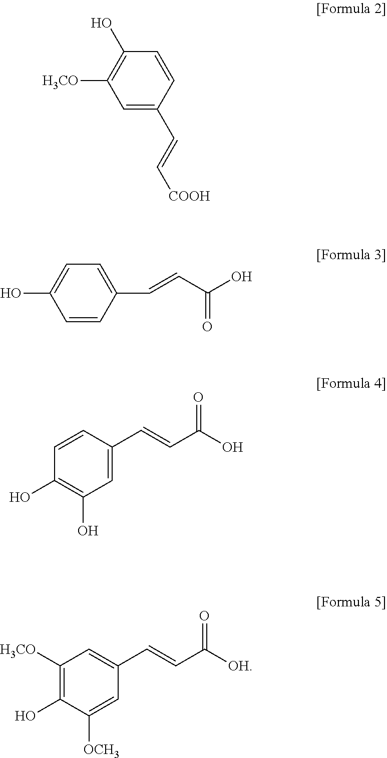 [SMILES: COc1cc(/C=C/C(=O)O)ccc1O ; O=C(O)/C=C/c1ccc(O)cc1 ; O=C(O)/C=C/c1ccc(O)c(O)c1 ; COc1cc(/C=C/C(=O)O)cc(OC)c1O]

3. The pharmaceutical composition of claim 1, wherein the ferulic acid or analog thereof increases the expression of keratin 6A protein.

4. The pharmaceutical composition of claim 1, wherein the skin diseases caused by genetic mutation include suppressing the expression of a gene encoding at least one keratin selected from the group consisting of keratin 1, keratin 2, keratin 5, keratin 6A, keratin 6B, keratin 9, keratin 14, keratin 10, keratin 16 and keratin 17.

5. The pharmaceutical composition of claim 1, wherein the ferulic acid or analog thereof regulates the expression of beta-catenin protein such that the ferulic acid or analog thereof suppresses the expression of beta-catenin protein in the epidermal layer of the skin, and induces the expression of beta-catenin protein in the dermal layer of the skin.

6. The pharmaceutical composition of claim 1, wherein the skin diseases caused by genetic mutation are at least one selected from the group consisting of psoriasis, epidermolysis bullosa (EB), Dermatopathia pigmentosa reticularis (DPR), Dowling-Degos Disease (DDD), Epidermolytic Hyperkeratosis (EHK), Bullous Congenital ichthyosiform erythroderma (BCIE), Ichthyosis Bullosa of Siemens (IBS), Palmoplantar keratoderma (PPK), Epidermolytic Palmoplantar keratoderma (EPPK), Pachyonychia congenital, Steatocystoma multiplex and skin cancer.

7. The pharmaceutical composition of claim 1, wherein the pharmaceutical composition is administered via an oral, nasal, topical, transdermal or parenteral route.

8. A cosmetic composition for preventing or improving skin diseases caused by genetic mutation comprising ferulic acid of the following Formula 1, an analog thereof or a cosmetically acceptable salt thereof: 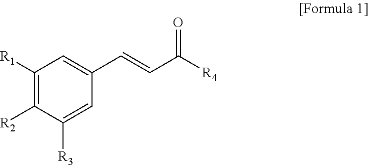
wherein R₁ is H, F, Br, Cl or I;
R₂ is OH, SH, NH, SeH, F, Br, Cl or I;
R₃ is OCH₃, CH₃, OC₂H₅ or OC₃H₇; and
R₄ is COOH, PO₄, CH₃, NH₃ or NHCOCH₃.

9. The cosmetic composition of claim 8, wherein a compound represented by the Formula 1 is any one selected from the group consisting of Formulae 2 to 5:

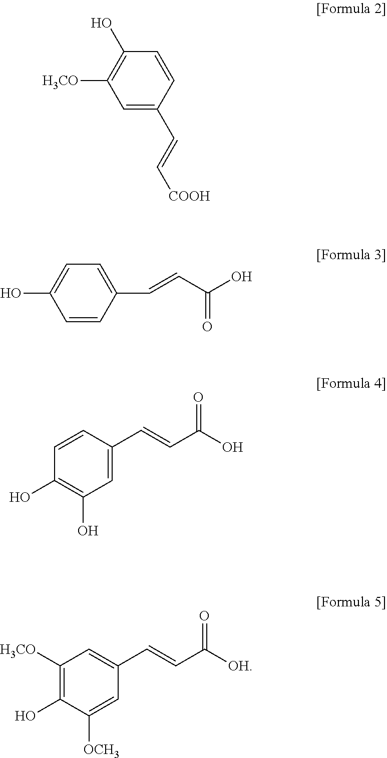 [SMILES: COc1cc(/C=C/C(=O)O)ccc1O ; O=C(O)/C=C/c1ccc(O)cc1 ; O=C(O)/C=C/c1ccc(O)c(O)c1 ; COc1cc(/C=C/C(=O)O)cc(OC)c1O]

10. The cosmetic composition of claim 8, wherein the skin diseases caused by genetic mutation are at least one selected from the group consisting of psoriasis, epidermolysis bullosa (EB), Dermatopathia pigmentosa reticularis (DPR), Dowling-Degos Disease (DDD), Epidermolytic Hyperkeratosis (EHK), Bullous Congenital ichthyosiform erythroderma (BCIE), Ichthyosis Bullosa of Siemens (IBS), Palmoplantar keratoderma (PPK), Epidermolytic Palmoplantar keratoderma (EPPK), Pachyonychia congenital, Steatocystoma multiplex and skin cancer.

11. A food composition for preventing or improving skin diseases caused by genetic mutation comprising ferulic acid of the following Formula 1, an analog thereof or a food acceptable salt thereof: 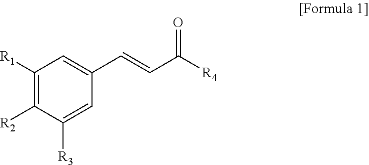
wherein R₁ is H, F, Br, Cl or I;
R₂ is OH, SH, NH, SeH, F, Br, Cl or I;
R₃ is OCH₃, CH₃, OC₂H₅ or OC₃H₇; and
R₄ is COOH, PO₄, CH₃, NH₃ or NHCOCH₃.

12. The food composition of claim 11, wherein a compound represented by the Formula 1 is at least one selected from the group consisting of the following formulae 2 to 5:

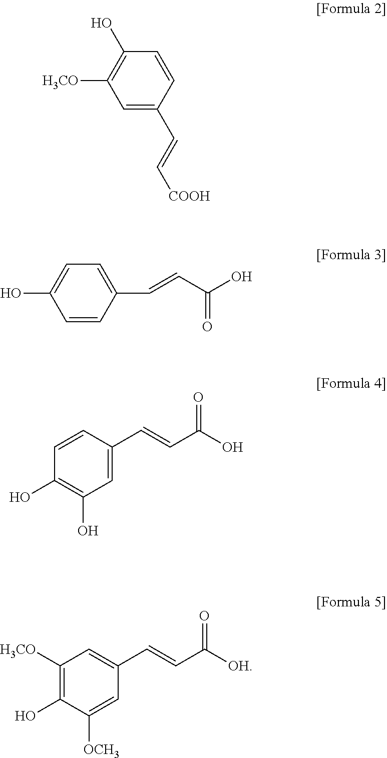 [SMILES: COc1cc(/C=C/C(=O)O)ccc1O ; O=C(O)/C=C/c1ccc(O)cc1 ; O=C(O)/C=C/c1ccc(O)c(O)c1 ; COc1cc(/C=C/C(=O)O)cc(OC)c1O]

13. The food composition of claim 11, wherein the skin diseases caused by genetic mutation are at least one selected from the group consisting of psoriasis, epidermolysis bullosa (EB), Dermatopathia pigmentosa reticularis (DPR), Dowling-Degos Disease (DDD), Epidermolytic Hyperkeratosis (EHK), Bullous Congenital ichthyosiform erythroderma (BCIE), Ichthyosis Bullosa of Siemens (IBS), Palmoplantar keratoderma (PPK), Epidermolytic Palmoplantar keratoderma (EPPK), Pachyonychia congenital, Steatocystoma multiplex and skin cancer.

14. The food composition of claim 11, wherein the food composition is a health functional food.

15. A method for preventing or treating skin diseases caused by genetic mutation, comprising administering to an individual the composition of claim 1.
